(19) United States
(12) Patent Application Publication
(10) Pub. No.: US 2007/0239063 A1
(43) Pub. Date: Oct. 11, 2007

(54) Portable electroretinograph with automated, flexible software

(76) Inventor: Kristina Narfstrom, Columbia, MO (US)

Correspondence Address: Medlen & Carroll, LLP, Suite 350, 101 Howard Street, San Francisco, CA, 94105, US

(21) Appl. No.: 11/701,439

(22) Filed: Feb. 1, 2007

Related U.S. Application Data
(60) Provisional application No. 60/764,181, filed on Feb. 1, 2006.

(51) Int. Cl. A61N 1/00

(52) U.S. Cl. 600/558

(57) ABSTRACT

The present invention relates to electroretinography (ERG) units and related methods of use. In particular, the present invention relates to electroretinography units used, for example, for evaluating the retinal function of a subject. The ERG unit of the present invention is portable and contains a compact flash card for flexible and automated ERG evaluation.

This application claims the benefit of U.S. Provisional Application 60/764,181, filed Feb. 1, 2006, and which is incorporated by reference herein in its entirety.

### FIELD OF THE INVENTION

The present invention relates to electroretinography (ERG) units and related methods of use. In particular, the present invention relates to electroretinography units used, for example, to provide a means to evaluate or for evaluation of retinal function of a subject.

### BACKGROUND OF THE INVENTION

Diagnosis of an eye disorder is initially based on the appearance of the eyes and on the symptoms that a subject is experiencing. A variety of tests can be carried out to confirm a problem or to determine the extent or severity of the disorder. Electroretinography is used to measure the electrical responses of various cell types in the retina, including, for example, the light-sensitive cells (rods and cones) and the bipolar cells. Electrodes are placed on the cornea and the skin near the eye. During a recording, a subject observes a standardized stimulus and the resulting signal is interpreted in terms of its amplitude (voltage) and time course. Stimuli include flashes (e.g., flash ERG) and reversing checkerboard patterns (e.g., pattern ERG). Applications are predominantly in ophthalmology, where the electroretinogram (ERG) is used for the diagnosis of various retinal diseases (e.g., Retinitis pigmentosa and related hereditary degenerations; Retinitis pigmentosa sine pigmento; Retinitis punctata albescens; Leber's congenital amaurosis; Choroideremia; Gyrate atrophy of the retina and choroid; Goldman-Favre syndrome; Congenital stationary night blindness; X-linked juvenile retinoschisis; Achromatopsia; Cone dystrophies; Disorders mimicking retinitis pigmentosa).

Presently available devices for conducting electroretinography (e.g., ERG units) tend to be large and cumbersome, and have several parts requiring assembly upon each use. In addition, presently available ERG units have a fixed software memory precluding software updates. What is needed are improved ERG units capable of easier use and program memory updating. In addition, what are needed are improved devices for evaluating the retinal function of a subject.

### SUMMARY OF THE INVENTION

The present invention relates to electroretinography (ERG) units and related methods of use. In particular, the present invention relates to electroretinography units used, for example, to provide a means to evaluate or for evaluation of retinal function of a subject.

In certain embodiments, the present invention provides an electroretinograph unit having therein a removable memory card, wherein the electroretinograph unit is configured to provide a means to evaluate or for evaluation of retinal function of a subject through utilization of electroretinograph-software stored in the removable memory card.

In certain embodiments, the removable memory card is a 64 Mb compact flash card. In certain embodiments, the removable memory card is selected from the group consisting of a secure digital card, a compact flash card, a memory stick, a multimedia card, a xD-picture card, and a smartmedia card. In other embodiments, the electroretinograph comprises a photostimulator.

In certain embodiments, the retinal function evaluation includes electroretinography to a weak flash in a dark-adapted eye of the subject; electroretinography to a strong flash in a dark-adapted eye of the subject; oscillatory potential measurement in an eye of the subject; electroretinography to a strong flash in a light-adapted eye of the subject; electroretinography to a rapidly repeated stimulus in an eye of the subject. In certain embodiments, the retinal function evaluation includes a user designed test of an eye of the subject.

In certain embodiments, the subject is a mammal. In certain embodiments, the subject is selected from the group consisting of a human, a cat, a dog, a pig, a horse, a mouse, a rat, and a rabbit. In certain embodiments, the electroretinograph unit obtains at least one set of data during the retinal function evaluation. In certain embodiments, the at least one set of data obtained during the retinal function evaluation is stored in the removable memory card.

In certain preferred embodiments, the present invention provides a method of evaluating the retinal function of a subject, comprising providing an electroretinograph unit having therein a removable memory card, wherein the electroretinograph unit is configured to evaluate the retinal function of a subject through utilization of electroretinograph-software stored in the removable memory card; and conducting an evaluation of the subject's retinal function with the electroretinograph unit.

In certain preferred embodiments, the present invention provides a method of diagnosing a retinal disease, comprising providing a subject suspected of having a retinal disorder, and an electroretinograph unit having therein a removable memory card, wherein the electroretinograph unit is configured to provide a means to evaluate or for evaluation of retinal function of a subject through utilization of electroretinograph-software stored in the removable memory card; conducting an evaluation of the subject's retinal function with the electroretinograph unit; and diagnosing the presence or absence of the retinal disorder based upon the conducted evaluation of the subject's retinal function. In certain embodiments, the retinal disorder is selected from the group consisting of Retinitis pigmentosa, Retinitis pigmentosa sine pigmento; Retinitis punctata albescens; Leber's congenital amaurosis; Choroideremia; Gyrate atrophy of the retina and choroid; Goldman-Favre syndrome; Congenital stationary night blindness; X-linked juvenile retinoschisis; Achromatopsia; Cone dystrophies; and disorders mimicking retinitis pigmentosa.

In certain preferred embodiments, the present invention provides a system comprising a removable memory card, and an electroretinograph unit having therein a dock for receiving the removable memory card, wherein the electroretinograph unit is configured to provide a means to evaluate or for evaluation of retinal function of a subject through utilization of electroretinograph-software stored in the removable memory card.

The ERG units of the present invention may be combined within various kit embodiments. For example, the present invention provides kits comprising, for example, an ERG unit of the present invention and a memory card (e.g., compact flash card) and/or a personal computer. In other kit embodiments, the present invention provides an ERG unit and an accessory agent (e.g., medication for subject in preparation for electroradiography procedure, electrodes for the subject's eyes, etc.). Additionally, the present invention contemplates kits comprising instructions (e.g., electroretinography instructions, pharmaceutical instructions) along with the ERG units of the present invention.

### BRIEF DESCRIPTION OF THE DRAWINGS

FIG. 1 shows an ERG unit of the present invention.

FIG. 2 shows an ERG unit of the present invention.

FIG. 3 shows an image of an ERG unit of the present invention.

FIG. 4 shows an ERG unit of the present invention attached onto a tri-pod.

FIG. 5 shows an ERG unit of the present invention.

FIGS. 6 and 7 show a dog and cat, respectively, undergoing a retinal function evaluation with an ERG unit of the present invention.

FIG. 8 provides thumbnail ERG recordings taken with an ERG unit of the present invention.

### DEFINITIONS

To facilitate an understanding of the invention, a number of terms are defined below.

As used herein, the term “operating software” refers to software designed to control the hardware of an ERG unit of the present invention in order to allow users and application programs (e.g., electroretinograph-software stored in a memory card) to make use of it.

As used herein, the terms “subject” refers to any animal, such as a mammal like a dog, cat, horse, pig, rat, rabbit, livestock, and preferably a human. Specific examples of “subjects” and “patients” include, but are not limited to, individuals requiring a retinal function evaluation.

As used herein, the term “memory card” refers to an electronic device that stores data (e.g., data sets obtained from an ERG unit) and software (e.g., electroretinograph-software utilized by an ERG unit to perform electroretinography. Examples of memory cards include, but are not limited to, a secure digital card, compact flash card, memory stick, multimedia card, xD-picture card, smartmedia card. As used in the present invention, memory cards are not limited to a particular memory size (e.g., 1 MB, 50 MB, 100 MB, 1 GB, 100 GB, 1000 GB).

As used herein, the term “electroretinography” refers to a test wherein an electrode is placed on the cornea of a subject's eye to measure the electrical response of the rods and cones in the retina. It is useful, for example, in the evaluation of hereditary and acquired disorders of the retina. A normal test will show the appropriate changes in electroretinographic wave-form in response to changes in light stimulation of the retina, such as, for example, an increased intensity of light stimulation or an increase in the frequency of the light stimulation. Abnormal results can indicate, for example, generalized retinal changes, such as hereditary retinal degeneration, retinal detachment or toxic retinopathy.

As used herein, the terms “retinal disorder” or “retinal disease” or similar term refers to a disorder of the retina. Examples include, but are not limited to, Retinitis pigmentosa and related hereditary degenerations; Retinitis pigmentosa sine pigmento; Retinitis punctata albescens; Leber's congenital amaurosis; Choroideremia; Gyrate atrophy of the retina and choroid; Goldman-Favre syndrome; Congenital stationary night blindness; X-linked juvenile retinoschisis; Achromatopsia; Cone dystrophies; Disorders mimicking retinitis pigmentosa.

### DETAILED DESCRIPTION

The present invention relates to electroretinography (ERG) units and related methods of use. In particular, the present invention relates to electroretinography units used, for example, to provide a means to evaluate or for evaluation of retinal function of a subject. The illustrated and preferred embodiments describe the ERG units of the present invention in terms of opthalmological applications (e.g., evaluating retinal function). However, it should be appreciated that the devices are not limited to opthalmological applications.

When the retina of the eye is stimulated by a flash of light, there is a characteristic sequence of electrical potentials generated within the retina. The clinical electroretinogram (ERG) is a recording of these potentials as detected between an electrode on or close to the cornea and an indifferent electrode placed on the forehead, cheek or ear lobe. The corneal electrode may be in the form of a contact lens with a steel or silver wire embedded in the inner surface, or it may be a piece of gold leaf tucked under the lower lid close to the cornea. A skin electrode on the lower lid of the eye can be used instead of the corneal electrodes but the result is less satisfactory.

It is common to perform the test with the eyes dark adapted (e.g., after spending several minutes in complete darkness). Under these conditions the ERG response is relatively large (e.g., 200 microvolts) and produces a wave with several distinct components covering about 200 milliseconds, but the components used for clinical diagnosis (known as a and b-waves) occur during, for example, 10-80 milliseconds.

The flash of light is normally provided from a stroboscopic flash unit and it is repeated a number of times so that the resulting electrical response can be fed to a signal averager to improve the signal-to-noise ratio and to reduce artifacts such as the blink response. Thus the recording apparatus will include a preamplifier capable of dealing with input signals between, for example, 0 and 1.5 mV, with frequency response from 0.3 to 300 Hz, a signal averager, and a display or recording device.

The ERG apparatus is normally found in the eye clinic or electrophysiological laboratory or it is part of a generalized instrument which may also be used for the electro-oculogram (EOG), electromyogram (EMG), possibly the visual evoked response (VER), and the electroencephalogram (EEG). The ERG is commonly used to assist in making a diagnosis of the various inherited disorders of the eye.

The ERG units of the present invention provide a convenient means to evaluate retinal function of a subject. The battery operation, handheld size, and integral photostimulator makes the ERG units of the present invention particularly useful in both clinical and laboratory settings as well as testing in the field. In addition, battery operation frees the ERG units of the present invention from any connections to AC power sources thereby eliminating a shock hazard and troublesome sources of line noise which plague this type of highly sensitive voltage measuring instrument.

The ERG unit devices of the present invention provide numerous advantages over prior art ERG devices. In particular, the ERG units of the present represent an improvement over the Mjolner ERG unit (Globaleye). For example, the ERG units of the present invention incorporate a “mini-Ganzfeld” (full field) photostimulator. In addition, the ERG units of the present invention utilize a memory card (e.g., compact flash card) for performance software and as a means for storing obtained data sets. The Mjolner ERG unit has a fixed memory that is incapable of performance software upgrading. Furthermore, the memory card utilized in the ERG units of the present invention can be removed and installed into a user's personal computer for analysis of obtained data sets.

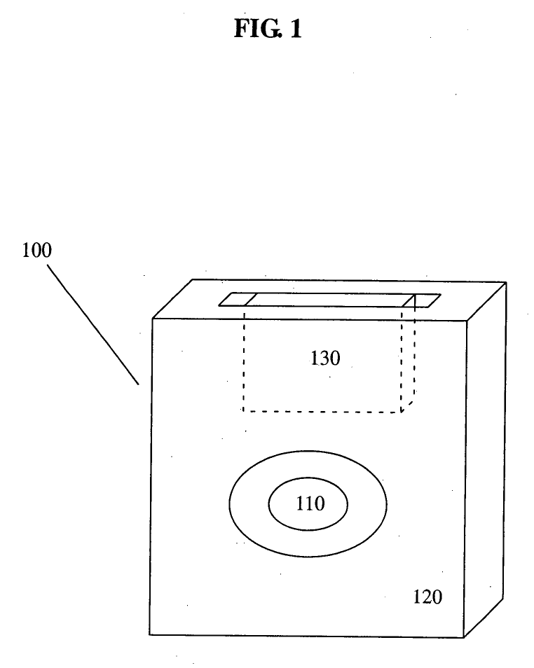

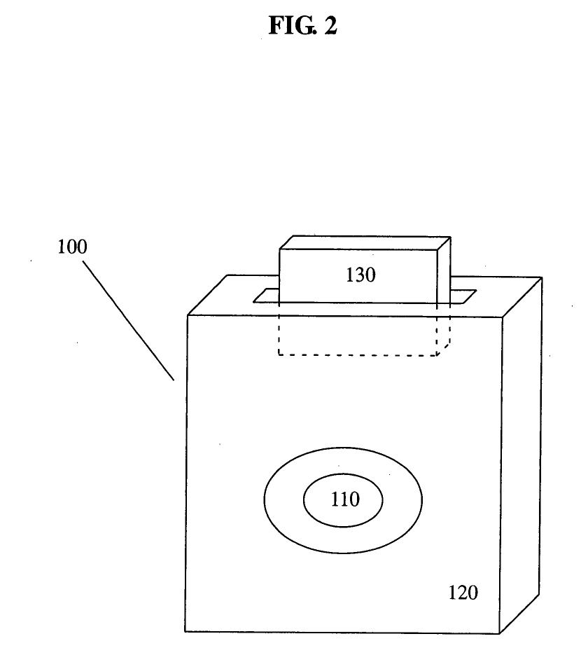

FIGS. 1 and 2 show a schematic image of an ERG unit 100 of the present invention. The present invention is not limited to a particular type or kind of ERG unit 100. The ERG unit 100 generally comprises a photostimulator 110, a central housing 120, and a memory card 130. The ERG unit 100 is designed for use with any kind of subject (e.g., human, dog, cat, monkey, large mammal, small mammal). The ERG unit 100 is not limited to a particular size. In preferred embodiments, the ERG unit 100 weighs less than 5 pounds (e.g., less than 4.5 pounds, less than 3 pounds, less than 2 pounds, less than 1.5 pounds, less than 1 pound). In preferred embodiments, the ERG unit 100 weighs less than 1.5 pounds. In preferred embodiments, the ERG unit 100 is designed for portable use described in more detail below). In preferred embodiments, the ERG unit 100 is designed for hand-held use (described in more detail below). In preferred embodiments, the ERG unit 100 is designed to provide a means to evaluate or for evaluation of retinal function of a subject (described in more detail below).

Still referring to FIGS. 1 and 2, the central housing 120 has a central housing front face 122, a central housing back face 124, a central housing top end 126, a central housing bottom end 128, and two central housing sides 129. The central housing 120 is not limited to a particular location for engaging the photostimulator 110. In preferred embodiments, the photostimulator 110 engages the central housing 120 along the central housing back face 124. The central housing 120 is not limited to a particular location for receiving the memory card 130. In preferred embodiments, the central housing 120 receives the memory card 130 along the central housing top end 126. The central housing 120 is not limited to particular size dimensions. The central housing 120 is not limited to a particular shape.

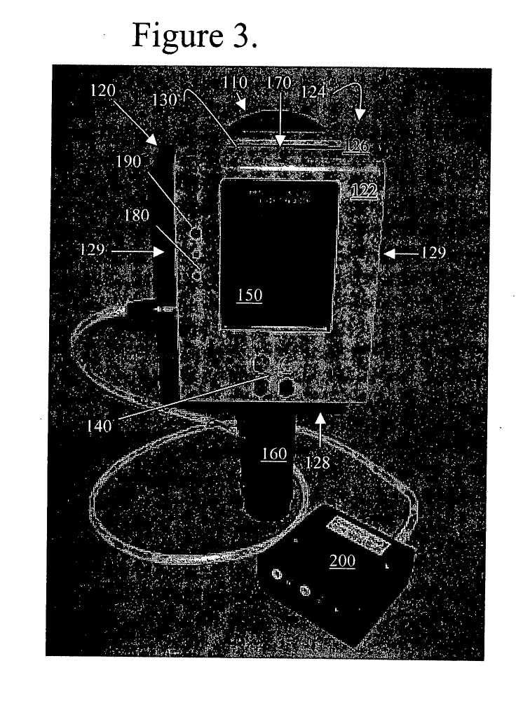

Still referring to FIGS. 1 and 2, the central housing 120 has therein an operating system. The central housing 120 is not limited to a particular type of operating system 120. In preferred embodiments, the operating system 120 is designed to operate the photostimulator 110, operate software stored in the memory card 130, process images received from the photostimulator 110, and provide images received from the photostimulator 110 to the memory card 130. FIG. 3 presents an image of an ERG unit 100 with a photostimulator 110 and a central housing 120. As shown in FIG. 2, the central housing 120 has a keyboard 140, a display panel 150, a hand-held stand 160, a memory card slot 170, an assortment of display lights 180, a photosensor 190, and an electrode pod 200.

Still referring to FIG. 3, the central housing 120 has a keyboard 140. The keyboard 140 is positioned on the outside of the central housing 120. The keyboard 140 is not limited to a particular position location along on the outside of the central housing 120. In preferred embodiments, the keyboard 140 is positioned along the central housing front face 122. The central housing 120 is not limited to a particular type of keyboard 140 (e.g., a traditional alphabet keyboard, a numerical keyboard, assorted buttons). In preferred embodiments, the keyboard 140 has an arrangement of keys. The keyboard 140 is not limited to a particular number of keys (e.g., 100, 50, 20, 10, 5, 4, 3, 2 . . . ). In preferred embodiments, the keyboard 140 has four keys. The keyboard 140 is designed to interact with software stored in a memory card 130 positioned within the central housing 120 (described in more detail below). In preferred embodiments, the function for each key within the keyboard 140 is determined by software stored in a memory card 130 positioned within the central housing 120. The keyboard is not limited to a particular set of functions. In preferred embodiments, the keyboard 140 controls operation of the photostimulator 110, controls which type of memory card 130 based software to run, and controls the processing of images obtained from the photostimulator 110.

Still referring to FIG. 3, the central housing 120 has a display panel 150. The display panel 150 is positioned on the outside of the central housing 120. The display panel 150 is not limited to a particular position location along on the outside of the central housing 120. In preferred embodiments, the display panel 150 is positioned along the central housing front face 122. The display panel 150 is not limited to particular size dimensions. The display panel 150 can either be black and white, color, or a mixture thereof. The display panel 150 can be configured to present images (e.g., ERG images), display text, or mixtures thereof. The display panel 150 is not limited to a particular resolution. The brightness of the display panel 150 can be adjusted to a preferred level. In preferred embodiments, the display panel 150 is a 75×53 mm, color TFT display monitor. In preferred embodiments, the background illumination of the display panel 150 can be set to one of three levels depending upon the room lightning level (e.g., the brightness of the display panel 150 can be pre-adjusted to a level suitable for a dark room or a well lighted room). In preferred embodiments, the display panel 150 adjusts the screen brightness based upon information obtained from the photosensor 190 (described in more detail below).

Still referring to FIG. 3, the central housing 120 has a hand-held stand 160. The hand-held stand 160 is positioned on the outside of the central housing 120. The hand-held stand 160 is not limited to a particular position location along on the outside of the central housing 120. In preferred embodiments, the hand-held stand 160 is positioned along the central housing bottom end 128. The hand-held stand 160 is not limited to a particular length or width. In preferred embodiments, the length and width of the hand-held stand 160 is such that a user may comfortably grasp and control the ERG unit 100. In some embodiments, the hand-held stand 160 has a trigger for operating the ERG unit 100. In such embodiments, the trigger functions in unison with the keyboard 140. In some embodiments, the hand-held stand 160 is configured for attachment with a tri-pod. In some embodiments, the hand-held stand 160 is adjustable such that it can pivot to accommodate the positioning of the ERG unit 100 into a preferred angle.

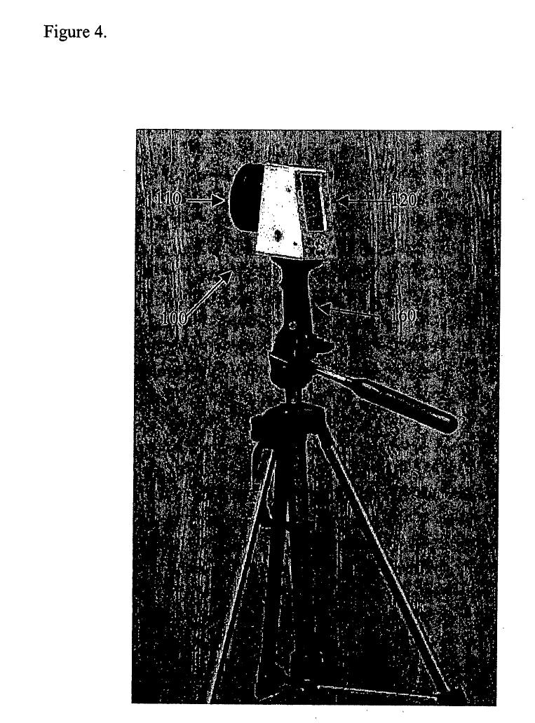

FIG. 4 shows an ERG unit 100 of the present invention attached onto a tri-pod. The ERG unit 100 has a photostimulator 110, and a central housing 120 with a hand-held stand 160. As shown in FIG. 4, the tri-pod is attached with the ERG unit 100 at the hand-held stand 160.

Referring again to FIG. 3, the central housing 120 has a memory card dock 170. The memory card dock 170 is positioned on the outside of the central housing 120. The memory card dock 170 is not limited to a particular position location along on the outside of the central housing 120. In preferred embodiments, the memory card dock 170 is positioned along the central housing top end 128. In preferred embodiments, the memory card dock 170 is positioned such that a memory card 130 is accessible from the outside of the central housing 120. The memory card dock 170 is not limited to receiving a particular type of memory card 130. The memory card dock 170 is not limited to particular size dimensions. In preferred embodiments, as the memory card dock 170 is not limited to a particular manner of receiving and ejecting a memory card 130. In preferred embodiments, the memory card dock 170 has therein a spring-lock mechanism for receiving and ejecting a memory card 130.

Still referring to FIG. 3, the central housing 120 has an assortment of display lights 180. The display lights 180 are positioned on the outside of the central housing 120. The display lights 180 are not limited to a particular position location along on the outside of the central housing 120. In preferred embodiments, the display lights 180 are positioned along the central housing front face 122. The central housing 120 is not limited to a particular number of display lights 180 (e.g., 1, 2, 3, 4, 5, 10, 20, 50 . . . ). In preferred embodiments, the central housing 120 has three display lights 180. The display lights 180 are not limited to a particular color. In preferred embodiments, the operation of the display lights 180 is controlled by software within a memory card 130 positioned within the memory card dock 170.

Still referring to FIG. 3, the central housing 120 has a photosensor 190. The photosensor 190 is positioned on the outside of the central housing 120. The photosensor 190 is not limited to a particular position location along on the outside of the central housing 120. In preferred embodiments, the photosensor 190 is positioned along the central housing front face 122. The central housing 120 is not limited to a particular type of photosensor 190. In preferred embodiments, the photosensor 190 is designed to detect the amount of light in a setting (e.g., room) and provide that information to operating system of the central housing 120, which in return provides such information to the software stored in the memory card 130. In preferred embodiments, the display panel 150 adjusts its level of illuminence based upon setting brightness information obtained with the photosensor 190.

Still referring to FIG. 3, the central housing 120 has therein an electrode pod 200. The electrode pod 200 has therein, for example, electrode signal conditioning circuitry and an amplifier. The electrode pod 200 is positioned separate from the central housing 120 and is connected with the central housing 120 via an input/output port (described in more detail below). The central housing 120 is not limited to a particular kind of electrode pod 200. In preferred embodiments, the electrode pod 200 is provides an assortment of ports for an assortment of electrodes. The electrode pod 200 is not limited to interfacing with a particular type of electrode. In preferred embodiments, the electrode pod 200 is designed for electroretinograph examination (e.g., electrodes that contact the cornea or nearby bulbar conjunctiva; lens electrodes, conjunctival loop electrodes, reference electrodes, ground electrodes, skin reference electrodes, etc). In preferred embodiments, the electrode pod 200 is designed to interface with electrodes in full compliance with the standards for clinical electroretinography established by the International Society for Clinical Electrophysiology of Vision (see, e.g., Marmor, M. F., et al., Documenta Ophthalmologica 108:107-114 (2004); herein incorporated by reference in its entirety). In preferred embodiments, information collected with an electrode (e.g., retinal function information) is collected with the electrode pod 200, relayed to the central housing 120, and relayed to software stored in the memory card 130.

Still referring to FIG. 3, the central housing 120 has therein an amplifier. The central housing 120 is not limited to a particular type of amplifier. In preferred embodiments, the specifications of the amplifier is include a 2 channel, 5,000 gain, noise, <2 uV p-p (0.3-300 Hz). In preferred embodiments, the amplifier interfaces with the operating system and software within a memory card 130 positioned within the central housing 120 through, for example, an analog to digital converter. In preferred embodiments, volume of the amplifier is adjustable.

Still referring to FIG. 3, the central housing 120 has therein a battery. The central housing is not limited to a particular type of battery. In preferred embodiments, the battery is rechargeable. In preferred embodiments, the specifications of the battery are 3.6 V, 9 Ah. In preferred embodiments, the battery is configured to provide 10 hours of continuous operation of the ERG unit 100. In preferred embodiments, the battery is connected with a battery recharge port (described in more detail below).

Still referring to FIG. 3, the central housing 120 has therein various input/output ports. The input/output ports are positioned on the outside of the central housing 120. The input/output ports are not limited to a particular position location along on the outside of the central housing 120. In preferred embodiments, the input/output ports are positioned along the central housing sides 129. The central housing 120 is not limited to particular types of input/output ports. In preferred embodiments, the central housing 120 has a port for interfacing with a computer (e.g., a USB connection port). In preferred embodiments, the central housing 120 has a port for battery recharge. In preferred embodiments, the central housing 120 has an electrode pod port.

Still referring to FIGS. 1 and 2, show the ERG unit 100 with a memory card 130. FIG. 1 shows an ERG unit 100 with a memory card positioned within the central housing 120 (e.g., the memory card 130 is shown with dotted lines). FIG. 2 shows an ERG unit 100 with a memory card 130 in an ejected positioned. The ERG unit 100 is not limited to a particular kind of memory card 130 (e.g., secure digital card, compact flash card, memory stick, multimedia card, xD-picture card, smartmedia card). The memory card 130 is not limited to a particular amount of memory (e.g., 1 MB, 50 MB, 100 MB, 1 GB, 100 GB, 1000 GB). In preferred embodiments, the memory card 130 is a compact flash card with any desired amount of memory (e.g., 64 MB, 0.1 GB, 1 GB, 100 GB, 1000 GB, etc). In preferred embodiments, the ERG unit 100 is configured to provide a means to evaluate or for evaluation of retinal function of a subject through utilization of electroretinograph-software stored on the memory card 130. The memory card 130 is not limited to particular types of electroretinograph-software. In preferred embodiments, the memory card 130 contains electroretinograph-software designed for, for example, running tests and protocols with the ERG units 100 (e.g., electroretinography related tests and protocols) (e.g., electroretinography to a weak flash in a dark-adapted eye of a subject, electroretinography to a strong flash in a dark-adapted eye of said subject, evaluation of oscillatory potential measurement in an eye of a subject, electroretinography to a strong flash in a light-adapted eye of a subject, electroretinography to a rapidly repeated stimulus in an eye of a subject, or a user designed evaluation), perform data storage with the ERG units 100 (e.g., storing data sets obtained from electroretinography related tests), perform data analysis (e.g., interpret and analyze data sets obtained from electroretinography related tests, printing data sets), and perform impedence testing and baseline evaluation of the ERG unit 100 (e.g., detecting the correct set-up of the electrodes with a subject, detecting the correct function of the ERG units 100, and detecting the level of noise within the system). In preferred embodiments, the electroretinograph-software contained in the memory card 130 is easily upgradable (e.g., with any personal computer), thereby allowing easy upgrading of the ERG units 100. In preferred embodiments, the memory card 130 contains electroretinograph-software for ERG unit 100 performance of retinal function evaluation in compliance with the standards for clinical electroretinography established by the International Society for Clinical Electrophysiology of Vision (see, e.g., Marmor, M. F., et al., Documenta Ophthalmologica 108:107-114 (2004); herein incorporated by reference in its entirety). In preferred embodiments, the memory card 130 contains software designed for storage and retrieval of data sets obtained with the EGR unit 100.

Still referring to FIGS. 1 and 2, the ERG unit 100 is not limited to a particular type of photostimulator 110 (e.g., a Ganzfeld Stimulator). The photostimulator 110 is designed to provide light stimulation to a subject's retina for purposes of measuring the retinal electrical activity. In preferred embodiments, the photostimulator 110 is designed to provide light stimulation to a subject's retina in compliance with the standards for clinical electroretinography established by the International Society for Clinical Electrophysiology of Vision (see, e.g., Marmor, M. F., et al., Documenta Ophthalmologica 108:107-114 (2004); herein incorporated by reference in its entirety). In preferred embodiments, the photostimulator 110 is a Ganzfeld type 76 mm diameter Flash Dome with 55 mm aperture. In preferred embodiments, the photostimulator 110 is designed to uniformly illuminate a subject's eye (e.g., retina) with an intensity from 1 mcd.s/m² to 50 cd.s/m². In preferred embodiments, the photostimulator 110 is designed to uniformly illuminate a subject's eye (e.g., retina) with an intensity from 10 mcd.s/m² to 30 cd.s/m².

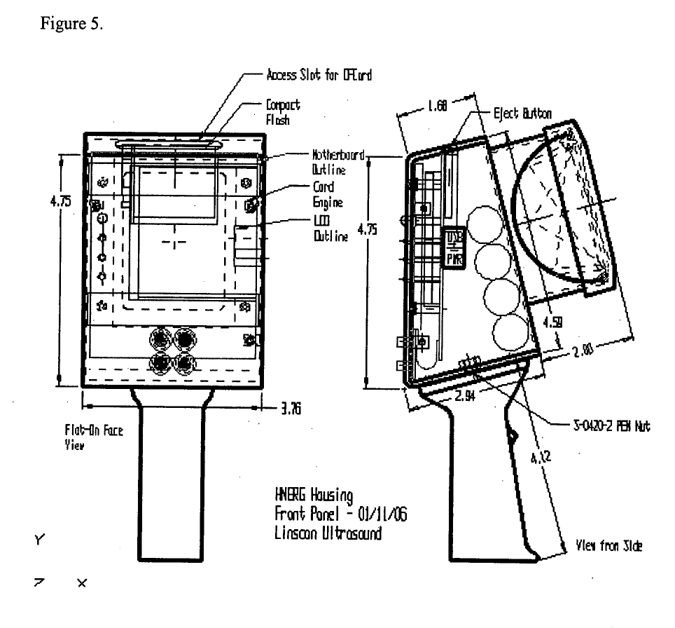

FIG. 5 shows an ERG unit embodiment of the present invention. As shown, the ERG unit has a photostimulator, and a central housing with a hand-held stand.

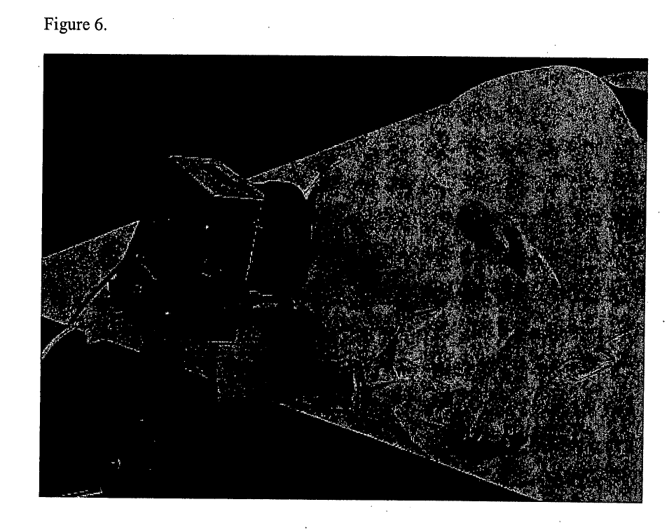

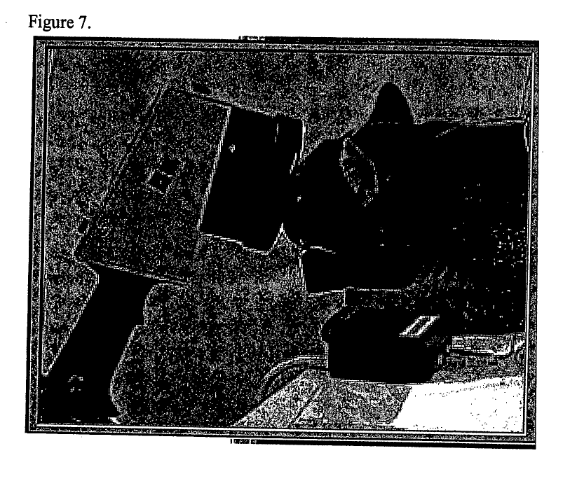

The ERG units of the present invention may be used in any medical technique involving the evaluation of a subject's retinal function. FIGS. 6 and 7 show a dog and cat, respectively, undergoing a retinal function evaluation with an ERG unit of the present invention. As shown in FIGS. 6 and 7, the ERG unit has a photostimulator, a central housing, and a memory card (not shown) positioned within the central housing. In such methods, an ERG unit is provided having therein a removable memory card, wherein the electroretinograph unit is configured to evaluate the retinal function of a subject through utilization of electroretinograph-software stored in the removable memory card, and conducting an evaluation of the subject's retinal function with the electroretinograph unit. In other preferred embodiments, the present invention provides a method of diagnosing a retinal disease, comprising providing a subject suspected of having a retinal disorder, and an electroretinograph unit having therein a removable memory card, wherein the electroretinograph unit is configured to evaluate the retinal function of a subject through utilization of electroretinograph-software stored in the removable memory card; conducting an evaluation of the subject's retinal function with the electroretinograph unit; and diagnosing the presence or absence of the retinal disorder based upon the conducted evaluation of the subject's retinal function. The present invention is not limited to the diagnosis of a particular retinal disorder (e.g., Retinitis pigmentosa, Retinitis pigmentosa sine pigmento; Retinitis punctata albescens; Leber's congenital amaurosis; Choroideremia; Gyrate atrophy of the retina and choroid; Goldman-Favre syndrome; Congenital stationary night blindness; X-linked juvenile retinoschisis; Achromatopsia; Cone dystrophies; and disorders mimicking retinitis pigmentosa). It is understood that one skilled in the art (e.g., a veterinarian, a physician) is able to diagnose a retinal function disorder through conducting electroretinograph examinations of a subject.

The ERG units of the present invention may be combined within various kit embodiments. For example, the present invention provides kits comprising, for example, an ERG unit of the present invention and a memory card (e.g., compact flash card) and/or a personal computer. In other kit embodiments, the present invention provides an ERG unit and an accessory agent (e.g., medication for subject in preparation for electroretinography procedure, electrodes for the subject's eyes, etc.). Additionally, the present invention contemplates kits comprising instructions (e.g., electroretinography instructions, pharmaceutical instructions) along with the ERG units of the present invention.

### EXAMPLES

### Example 1

This example describes various protocols which may be employed with the ERG units of the present invention.

| | | | | | | |
|---|---|---|---|---|---|---|
| | | | Number | | | |
| | | Flash | of | | Time | Elapsed |
| | ERG Test | Intensity | Flashes | Interval | Req. | Time |
| Step | Sessions | mcd · s/m² | Avg'd | Seconds | Seconds | Seconds |

No. 1- Protocol Type - ISCEV - Dark adapted Patient.
Room Lights are turned off when starting the Session (S0)

| | | | | | | |
|---|---|---|---|---|---|---|
| S0 | Dark | | | | Not | 0 |
| | Adaptation | | | | Spec- | |
| | | | | | ified | |
| S1 | Rods | 10 | 10 | 2 | 20 | 20 |
| S2 | Delay | | | 2 | 2 | 28 |
| S3 | Std. Rods | 3000 | 4 | 10 | 40 | 68 |
| | & Cones | | | | | |
| S4 | Delay | | | | 30 | 98 |
| S5 | Hi-Int. | 10000 | 4 | 20 | 80 | 178 |
| | Rods & | | | | | |
| | Cones | | | | | |
| S6 | Cones | 10000 | BG. | | 600 | 778 |
| | with BG | | | | | |
| | Adaptation | | | | | |
| S7 | Cones w/BG | 3000 | 32 | 0.5 | 16 | 794 |
| S7 | Delay | | | | 2 | 796 |
| S8 | HiCones | 10000 | 32 | 0.5 | 16 | 812 |
| | w/BG | | | | | |
| S8 | Delay | | | | 2 | 814 |
| S9 | Std. | 3000 | 128 | 0.032 | 4.224 | 818.2 |
| | Flicker | | | | | |
| | w/BG | | | | | |
| S10 | Delay | | | | 2 | 820.2 |
| S11 | Hi-Int. | 10000 | 128 | 0.032 | 4.224 | 824.4 |
| | Flicker | | | | | |
| | w/BG | | | | | |
| | Total | | | | | 13.74 |
| | Lapsed | | | | | |
| | time in | | | | | |
| | Minutes | | | | | |

No. 2 - Protocol Type - Dog Diagnostic - Light adapted patient.
Turn off the room light when starting the Session (S1)

| | | | | | | |
|---|---|---|---|---|---|---|
| S1 | 1^{st} Step | | | | 240 | 240 |
| | of Dark | | | | | |
| | Adaptation | | | | | |
| | Cycle | | | | | |
| S2 | Rods | 10 | 10 | 2 | 20 | 260 |
| | during | | | | | |
| | 1^{st} DA | | | | | |
| S3 | 2^{nd} DA | | | | 240 | 500 |
| S4 | Rods at | 10 | 10 | 2 | 20 | 520 |
| | end of | | | | | |
| | 2^{nd} DA | | | | | |
| S5 | 3^{rd} DA | | | | 240 | 760 |
| S6 | Rods at | 10 | 10 | 2 | 20 | 780 |
| | end of | | | | | |
| | 3^{rd} DA | | | | | |
| S7 | 4^{th} DA | | | | 240 | 1020 |
| S8 | Rods at | 10 | 10 | 2 | 20 | 1040 |
| | end of | | | | | |
| | 4^{th} DA | | | | | |
| S9 | 5^{th} DA | | | | 240 | 1280 |
| S10 | Rods at | 10 | 10 | 2 | 20 | 1300 |
| | end of | | | | | |
| | 5^{th} DA | | | | | |
| S11 | Delay | | | | 2 | 1302 |
| S12 | Std. Rods | 3000 | 4 | 10 | 40 | 1342 |
| | & Cones | | | | | |
| S13 | Delay | | | | 30 | 1372 |
| S14 | Hi-Int. | 10000 | 4 | 20 | 80 | 1452 |
| | Rods | | | | | |
| | & Cones | | | | | |
| S15 | Cones BG | 3000 | BG | | 600 | 2052 |
| | Adaptation | | | | | |
| S15 | Cones w/BG | 3000 | 32 | 0.5 | 16 | 2068 |
| S16 | Delay | | | | 2 | 2070 |
| S17 | Flicker | 3000 | 128 | 0.032 | 4.1 | 2074.1 |
| | w/BG | | | | | |
| | Total | | | | | 34.57 |
| | Lapsed | | | | | |
| | Time in | | | | | |
| | Minutes | | | | | |

No. 3 - Protocol Type - Short - Light adapted patient.
Turn off the room lights when starting the Session (S1)

| | | | | | | |
|---|---|---|---|---|---|---|
| S1 | Std. Rods | 3000 | 1 | | | 0 |
| | & Cones | | | | | |
| S2 | Dark | | | | 60 | 60 |
| | Adaptation | | | | | |
| S3 | Std. Rods | 3000 | 4 | 2 | 8 | 68 |
| | & Cones | | | | | |
| S4 | Dark | | | | 300 | 368 |
| | Adaptation | | | | | |
| S5 | Std. Rods | 3000 | 4 | 2 | 8 | 374 |
| | & Cones | | | | | |
| S6 | Dark | | | | 60 | 434 |
| | Adaptation | | | | | |
| S7 | Hi-Int. | 10000 | 4 | 20 | 80 | 514 |
| | Rods | | | | | |
| | & Cones | | | | | |
| 8 | Delay | | | | 2 | 516 |
| S9 | Std. | 3000 | 128 | 0.032 | 4.1 | 520.1 |
| | Flicker | | | | | |
| | Total | | | | | 8.66 |
| | Lapsed | | | | | |
| | Time in | | | | | |
| | Minutes | | | | | |

### Example 2

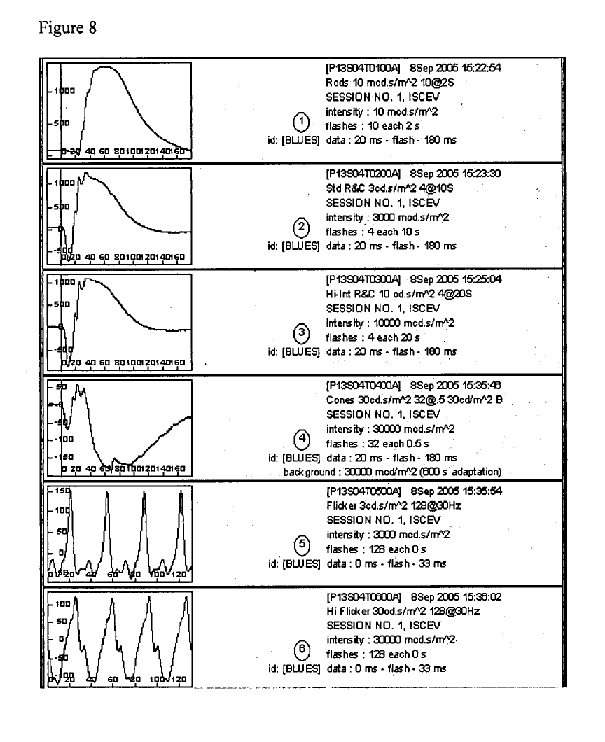

This example provides thumbnail ERG recordings taken with an ERG unit of the present invention. The sketch is presented in FIG. 8.

### Example 3

This example describes a recommended procedure for performing a scotopic ERG for a dog or cat with an ERG unit of the present invention.
1. Prepare the anesthetics to be used for the animals in the light and place the labeled syringes with needles on a tray in the dark room.
2. Dark adapt the animals, 20 min. to over night, depending on species.
3. Anesthetize the animal in the dark (red lights needed!) and dilate the pupil(s).
4. Bring the animal to the ERG room (dark!) after 10-15 minutes after giving deep sedation (Medetomidine+Ketamine) and on a table.
5. Put topical anesthesia into the eye(s) in which ERGs will be recorded.
6. Place the ground electrode (first) then the reference electrode; on top of the head (midline), and at the base of the ear, respectively.
7. Insert the lid speculum and place the corneal electrode after having placed a small drop of metylcellulose into the concave side of the contact lens.
8. Adjust the corneal contact lens so that it is centered on the cornea and so that the wire is positioned dorsally or temporally and fastened (if needed) with short piece of tape onto the skin (especially for dogs).
9. If the eye rotates downward significantly so that the pupil is not seen directly (may happen in dogs under anesthesia), stay sutures are placed at the conjunctival limbus, usually 3—one at the base of the corneal side of the 3^{rd} eyelid, one at the dorsal limbus and one at the ventral limbus, using silk sutures pulled together with hemostats that are taped to the skin positioning the bulb.
10. Run the ERG tests needed.
11. Give Antisedan to reverse the anaesthesia if Medetomidine/Ketamine has been used.
12. Keep the animal warm during the anesthesia and especially during the recovery period.

All publications and patents mentioned in the above specification are herein incorporated by reference. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

We claim:

1. An electroretinograph unit having therein a removable memory card, wherein said electroretinograph unit is configured to evaluate the retinal function of a subject through utilization of electroretinograph-software stored in said removable memory card.

2. The electroretinograph unit of claim 1, wherein said removable memory card is a 64 Mb compact flash card.

3. The electroretinograph unit of claim 1, wherein said removable memory card is selected from the group consisting of a secure digital card, a compact flash card, a memory stick, a multimedia card, a xD-picture card, and a smartmedia card.

4. The electroretinograph unit of claim 1, wherein said electroretinograph comprises a photostimulator.

5. The electroretinograph unit of claim 1, wherein said retinal function evaluation includes electroretinography to a weak flash in a dark-adapted eye of said subject.

6. The electroretinograph unit of claim 1, wherein said retinal function evaluation includes electroretinography to a strong flash in a dark-adapted eye of said subject.

7. The electroretinograph unit of claim 1, wherein said retinal function evaluation includes oscillatory potential measurement in an eye of said subject through utilization of said electroretinograph-software stored in said removable memory card.

8. The electroretinograph unit of claim 1, wherein said retinal function evaluation includes electroretinography to a strong flash in a light-adapted eye of said subject.

9. The electroretinograph unit of claim 1, wherein said retinal function evaluation includes electroretinography to a rapidly repeated stimulus in an eye of said subject.

10. The electroretinograph unit of claim 1, wherein said retinal function evaluation includes a user designed test of an eye of said subject.

11. The electroretinograph unit of claim 1, wherein said retinal function evaluation includes electroretinography in an eye of said subject.

12. The electroretinograph unit of claim 1, wherein said subject is a mammal.

13. The electroretinograph unit of claim 1, wherein said subject is selected from the group consisting of a human, a cat, a dog, a pig, a horse, a mouse, a rat, and a rabbit.

14. The electroretinograph unit of claim 1, wherein said electroretinograph unit obtains at least one set of data during said retinal function evaluation.

15. The electroretinograph unit of claim 14, wherein said at least one set of data obtained during said retinal function evaluation is stored in said removable memory card.

16. A method of evaluating the retinal function of a subject, comprising
providing an electroretinograph unit having therein a removable memory card, wherein said electroretinograph unit is configured to evaluate the retinal function of a subject through utilization of electroretinograph-software stored in said removable memory card;
conducting an evaluation of said subject's retinal function with said electroretinograph unit.

17. The method of claim 16, wherein said removable memory card is a 64 Mb compact flash card.

18. The method of claim 16, wherein said removable memory card is selected from the group consisting of a secure digital card, a compact flash card, a memory stick, a multimedia card, a xD-picture card, and a smartmedia card.

19. The method of claim 16, wherein said electroretinograph comprises a photostimulator.

20. The method of claim 16, wherein said retinal function evaluation includes electroretinography to a weak flash in a dark-adapted eye of said subject.

21. The method of claim 16, wherein said retinal function evaluation includes electroretinography to a strong flash in a dark-adapted eye of said subject.

22. The method of claim 16, wherein said retinal function evaluation includes oscillatory potential measurement in an eye of said subject through utilization of said electroretinograph-software stored in said removable memory card.

23. The method of claim 16, wherein said retinal function evaluation includes electroretinography to a strong flash in a light-adapted eye of said subject.

24. The method of claim 16, wherein said retinal function evaluation includes electroretinography to a rapidly repeated stimulus in an eye of said subject.

25. The method of claim 16, wherein said retinal function evaluation includes a user designed test of an eye of said subject.

26. The method of claim 16, wherein said retinal function evaluation includes electroretinography in an eye of said subject.

27. The method of claim 16, wherein said subject is a mammal.

28. The method of claim 16, wherein said subject is selected from the group consisting of a human, a cat, a dog, a pig, a horse, a mouse, a rat, and a rabbit.

29. The method of claim 16, wherein said electroretinograph unit obtains at least one set of data during said retinal function evaluation.

30. The method of claim 29, wherein said at least one set of data obtained during said retinal function evaluation is stored in said removable memory card.

31. A method of diagnosing a retinal disease, comprising
providing a subject suspected of having a retinal disorder, and an electroretinograph unit having therein a removable memory card, wherein said electroretinograph unit is configured to evaluate the retinal function of a subject through utilization of electroretinograph-software stored in said removable memory card;
conducting an evaluation of said subject's retinal function with said electroretinograph unit; and
diagnosing the presence or absence of said retinal disorder based upon said conducted evaluation of said subject's retinal function.

32. The method of claim 31, wherein said retinal disorder is selected from the group consisting of Retinitis pigmentosa, Retinitis pigmentosa sine pigmento; Retinitis punctata albescens; Leber's congenital amaurosis; Choroideremia; Gyrate atrophy of the retina and choroid; Goldman-Favre syndrome; Congenital stationary night blindness; X-linked juvenile retinoschisis; Achromatopsia; Cone dystrophies; and disorders mimicking retinitis pigmentosa.
